SEQUENCE LISTING (1 sequence)

SEQ ID NO: 1 - DNA, 24 bp, Artificial Sequence
  Oligonucleotide purified in Examples 1 and 2
tcgtcgtgttttctattttcgutt

(19) United States
(12) Patent Application Publication
(10) Pub. No.: US 2006/0035224 A1
(43) Pub. Date: Feb. 16, 2006

(54) Purification methods for oligonucleotides and their analogs

(76) Inventor: Jack T Johansen, Concord, MA (US)

Correspondence Address: MORGAN LEWIS & BOCKIUS LLP, 1111 PENNSYLVANIA AVENUE NW, WASHINGTON, DC, 20004, US

(21) Appl. No.: 10/508,799

(22) PCT Filed: Mar. 19, 2003
(86) PCT No.: PCT/GB03/01161

§ 371 Date: Sep. 21, 2004

Related U.S. Application Data
(60) Provisional application No. 60/367,060, filed on Mar. 21, 2002.

(51) Int. Cl. C12Q 1/68; C07H 21/04

(52) U.S. Cl. 435/6; 536/25.4

(57) ABSTRACT

The present invention discloses methods for separating oligonucleotides from impurities. In the methods of the invention, a target oligonucleotide, in a mixture comprising the target oligonucleotide and an impurity, is separated from the impurity using a titratable anion exchange composition. The target oligonucleotide is bound to the titratable anion exchange composition and an eluting solution which increases in pH over time is passed through the titratable anion exchange composition with the target oligonucleotide bound thereon. Preferably, the eluting solution does not substantially increase its salt concentration. The target oligonucleotide is eluted and thereby separated from the impurity which either elutes at a lower pH or a higher pH than the target oligonucleotide.

### BACKGROUND

Synthetic oligonucleotides have emerged as important biomolecules for a wide variety of applications. Such applications include the use of synthetic oligonucleotides as hybridization probes, linkers, primers for DNA sequencing, amplification reactions (e.g., polymerase chain reactions, reverse transcriptase reactions), potential therapeutics in antisense and related technology investigations and diagnostic tools for the detection of genetic and viral diseases. In addition, synthetic oligonucleotide analogs have received approval of the FDA for the treatment of CMV and at present, several oligonucleotide analogs are undergoing clinical trials.

Over the past several years, significant progress has been achieved in the synthesis of oligonucleotides and their analogs on a large scale (for example, in kilogram quantities). Much of this progress is due to the development of automated solid-phase DNA synthesizers which are capable of producing μmol to mol quantities of product in a single synthesis. In general, the synthesis of synthetic oligonucleotides is most frequently performed via a series of systematic reactions that result in the stepwise addition of specific oligonucleotides (protected at their 5′ hydroxy ends with a protecting group (e.g., dimethoxytrityl group (DMT)) to a nascent oligonucleotide chain attached to a solid phase support.

However, in spite of the technological advances in the production of synthetic oligonucleotides, during each stepwise addition of a monomer to the nascent oligonucleotide chain, approximately 1-2% of the coupling reactions fail (i.e., no monomer addition occurs). Consequently, the resulting products are generally a heterogeneous mixture of oligonucleotides of varying length, wherein the amount of undesired contaminants (e.g., failure sequences) is proportional to the length of the desired product and overall yield of the synthesis. As a result, this has led to an interest in developing manufacturing techniques for the purification of oligonucleotides.

Several techniques have been developed to separate or purify full length target oligonucleotides from failure sequences, including thin layer chromatography (TLC), gel electrophoresis (e.g., polyacrylamide gel electrophoresis (PAGE)) and liquid chromatographic techniques (e.g., high-performance liquid chromatography (HPLC)). For large scale separations of synthetic oligonucleotides, the two major chromatographic techniques that have been used are anion exchange chromatography and reverse-phase chromatography. Both of these conventional chromatographic separation methods, however, are not only time consuming and laborious, but also costly.

In the case of separating synthesized oligonucleotides using reverse-phase chromatography, a hydrophobic 5′ protecting group, e.g., a 5′-O-trityl group, is used to protect the 5′-hydroxyl group of the oligonucleotide during the coupling and oxidation steps. The hydrophobic 5′-protecting group can then be used to separate the full-length “trityl on” target oligonucleotide from shorter failure sequences which do not possess the hydrophobic 5′-protecting group (i.e., “trityl off” sequences). After separation, the trityl group can be cleaved from the target oligonucleotide with acid. While this is an effective approach, it is time consuming and laborious and requires subsequent hydrolysis and extraction steps to isolate the purified product.

In the case of separating synthesized oligonucleotides using anion exchange chromatography, an anion exchange composition containing fixed positive charges is used to bind the target oligonucleotide and failure sequences (both of which are polyanions). The strength of the binding of the target oligonucleotide and failure sequences to the anion exchange composition is directly proportional to their length. Thus, to elute the bound target oligonucleotide and failure sequences, a salt gradient is typically used to weaken the interaction of the oligonucleotides with the anion exchange composition. Since the strength of the binding is proportional to the length of the oligonucleotides, the shortest oligonucleotides elute first, while the longer oligonucleotides elute at higher salt concentrations. The use of a salt gradient in anion exchange separation of oligonucleotides is disadvantageous for a number of reasons. First, the desired eluted oligonucleotide typically has to be desalted prior to use in downstream applications. Consequently, this necessitates subsequent desalting steps which increase the time, labour and expense required for separation. Secondly, the use of high concentrations of salt in eluting solutions can also lead to increased corrosion of stainless steel parts (e.g., HPLC pumps, fittings, valves, columns, tubing) in the machinery used for anion exchange separations.

Thus, a need exists for improved methods of separation of oligonucleotides that overcomes or ameliorates the limitations and problems associated with current methods.

### SUMMARY OF THE INVENTION

The present invention is drawn to methods of separating oligonucleotides from impurities. In the methods of the invention, a target oligonucleotide, in a mixture comprising the target oligonucleotide and an impurity, is separated from the impurity using a titratable anion exchange composition. The target oligonucleotide is bound to the titratable anion exchange composition and an eluting solution which increases in pH over time is passed through the titratable anion exchange composition with the target oligonucleotide bound thereon. Preferably, the eluting solution does not substantially increase in salt concentration. The target oligonucleotide is then eluted, thereby separating it from the impurity which elutes at a different pH than the target oligonucleotide. Optionally, one or more washing steps can be performed prior to eluting the target oligonucleotide.

The methods of the invention can be used with a variety of titratable anion exchange compositions, including titratable anion exchange compositions which comprise a primary amine, a secondary amine or a tertiary amine. Suitable titratable anion exchange compositions include anion exchange compositions comprising polyimizadole, polyhistidine, polylysine or polyethyleneimine. In one embodiment, the titratable anion exchange composition is conjugated to a support, for example, a synthetic polymer support, such as silica gel, a polysaccharide, a polystyrene, especially a styrene-divinylbenzene copolymer, a polyethylene, a polypropylene, a polyacrylate, or an agarose, for example those agaroses available under the trade name Sepharose. Preferably, the titratable anion exchange composition is covalently bonded to a support, optionally via a linker group. The support may be a functionalised support. Examples of such functionalised supports are well known in the art, and include for example, the hydroxy or amino-functionalised supports, especially hydroxy or amino-functionalised polystyrene.

The methods of the invention can be used to separate a variety of oligonucleotides, including, for example, single-stranded and/or double-stranded nucleic acids. Such oligonucleotides include naturally-occurring oligonucleotides, such as deoxyribonucleic acids (DNA) and ribonucleic acids (RNA). Alternatively, the methods of the invention can be used to separate synthetic oligonucleotides, for example, phosphates, phosphorothioates, phosphorodithioates, methyl phosphonates, phosphoramidates and chimeras.

In one embodiment, the target oligonucleotide to be separated is 5′-O-protected (for example, with a 5′-O-trityl, such as a 5′-O-dimethoxytrityl, protecting group). Optionally, the 5′-O-protecting group is cleaved from the target oligonucleotide prior to elution, while the target oligonucleotide is bound to the titratable anion exchange composition. Cleavage of the 5′-O-trityl protecting group is achieved by passing through the titratable anion exchange composition with target oligonucleotide bound thereon, a sufficient amount of an acidic solution (e.g., a solution comprising aqueous acetic acid (e.g., 20%-80% v/v acetic acid)), prior to elution of the target oligonucleotide.

A variety of eluting solutions can be used to elute the target oligonucleotide provided that they increase pH. Separation of a target oligonucleotide from an impurity can be achieved without substantially increasing the salt concentration over time. Suitable eluting solutions are those that begin at a pH suitable for binding of the target oligonucleotide to the titratable anion exchange composition, and over time, increase in pH so that the titratable anion exchange composition cannot bind the target oligonucleotide. In one embodiment, the eluting solution is substantially free of metal salts.

The methods of the invention can be used to separate the target oligonucleotide from a variety of impurities. Such impurities include any composition which possesses a different molecular structure than the target oligonucleotide. In one embodiment, the impurity to be separated from the target oligonucleotide is one or more oligonucleotides having a shorter length than the target oligonucleotide (e.g., one or more failure sequences). In this embodiment, the impurity (i.e., one or more oligonucleotides having a shorter length than the target oligonucleotide) elutes at a lower pH than the target oligonucleotide. In another embodiment, the impurity to be separated from the target oligonucleotide comprises one or more salts, especially metal salts.

The methods of the invention can also be used to increase the concentration of a target oligonucleotide. In one embodiment, the target oligonucleotide is concentrated by eluting the target oligonucleotide with a volume of solution which is less than the volume that the target oligonucleotide was originally contained within.

The methods of the present invention are advantageous in that they avoid the more laborious and time-consuming conventional separation steps which are typically required to separate or purify oligonucleotides. In reducing the number of steps required to separate oligonucleotides, the present invention provides a rapid, simplified, more efficient and less expensive method for large-scale purification of oligonucleotides.

### DETAILED DESCRIPTION OF THE INVENTION

A description of preferred embodiments of the invention follows.

The present invention is drawn to methods of separating or purifying an oligonucleotide from an impurity. As used herein, the words “separating” and “purifying” are used interchangeably and refer to a process by which an oligonucleotide having a particular molecular structure is physically segregated from an impurity having a different molecular structure. Such segregation can be partial or complete.

An “oligonucleotide”, as defined herein, comprises an oligomer or polymer of nucleotides which are covalently linked by optionally modified phosphodiester bonds. Nucleotides have a common structure comprising an optionally modified phosphate group which is linked to a pentose which in turn is linked to an organic base. If the pentose is ribose, the nucleic acid is RNA and the nucleotides are ribonucleotides. If the pentose is 2′-deoxyribose, the nucleic acid is DNA and the nucleotides are deoxyribonucleotides. In aqueous solutions which have a pH greater than about 2, the very hydrophilic sugar-phosphate polymer backbone of a nucleic acid contributes one negative charge for each phosphodiester plus one or two negative charges for every terminal phosphomonoester. Thus, oligonucleotides are polyanions which possess a net negative charge which is approximately proportional to their length. A wide variety of bases may be attached to the pentose, but the five that predominate in naturally-occurring DNA and RNA are adenine (“A”), thymine (“T”, primarily in DNA), uracil (“U”, primarily in RNA), guanine (“G”), and cytosine (“C”).

As used herein, the terms “oligonucleotide” and “polynucleotide” are interchangeable and refer to a nucleotide multimer or oligomer having from a few, e.g., 2-20, to many, e.g., 20 to several hundred or more, for example up to 250, nucleotides. Oligonucleotides include double-stranded and single-stranded nucleic acids, e.g., single-stranded or double-stranded DNA, RNA or DNA-RNA hybrids. Oligonucleotides further include both naturally-occurring oligonucleotides and synthetic oligonucleotides.

Naturally-occurring oligonucleotides, as used herein, are nucleic acids that are found in an organism, for example, nucleic acids including but not limited to, genomic DNA, complimentary DNA (cDNA), chromosomal DNA, plasmid DNA, mRNA, tRNA and rRNA. Such naturally-occurring nucleic acids can also include altered nucleic acids, for example, naturally-occurring nucleic acids which contain additions, deletions or modifications of one or more nucleotides (e.g., polymorphic or allelic variants). Naturally-occurring oligonucleotides include nucleic acids which are isolated from an organism, for example, using methods described herein and/or other known methods.

Synthetic oligonucleotides are oligonucleotides which are prepared by artificial means, rather than isolated from an organism. For example, synthetic oligonucleotides include but are not limited to, oligonucleotides which are prepared on solid phases using well-known and/or commercially-available procedures (e.g., using an automated nucleic acid synthesizer or other chemical synthesis method). Synthetic oligonucleotides further include oligonucleotides which comprise one or more modified nucleotides. As used herein, a modified nucleotide is a nucleotide that has been structurally altered so that it differs from a naturally-occurring nucleotide. Such modified nucleotides include nucleotides which contains a modified sugar moiety, a modified phosphate moiety and/or a modified nucleobase.

Modification of the sugar moiety includes, but is not limited to, replacement of the ribose ring with a hexose, cyclopentyl or cyclohexyl ring. Alternatively, the D-ribose ring of a naturally-occurring nucleic acid can be replaced with an L-ribose ring or the β-anomer of a naturally-occurring nucleic acid can be replaced with the α-anomer.

Modified phosphate moieties include phosphorothioates, phosphorodithioates, methyl phosphonates, alkylphosphonates, alkylphosphonothioates, methyl phosphates, phosphoramidates, and the like, or combinations thereof. Oligonucleotides which comprise such modified phosphate linkages can have improved properties when compared to corresponding oligonucleotides comprising only phosphate diester linkages.

For example, oligonucleotides comprising modified linkages can have increased resistance to degradation by nucleases which may be present in an organism (e.g., when used in antisense applications). Modified nucleobases include 7-deazaguanine, 7-deaza-8-azaguanine, 5-propynylcytosine, 5-propynyluricil, 7-deazaadenine, 7-deaza-8-azaadenine, 7-deaza-6-oxopurine, 6-oxopurine, 3-deazaadenosine, 2-oxo-5-methylpyrimidine, 2-oxo-4-methylthio-5-methylpyrimidine, 2-thiocarbonyl-4-oxo-5-methylpyrimidine, 4-oxo-5-methylpyrimidine, 2-amino-purine, 5-fluorouricil, 2,6-diaminopurine, 8-aminopurine, 4triazolo-5-methylthymine, and 4-triazolo-5-methyluricil. Modified nucleobases can also include abasic moieties.

Methods for generating oligonucleotide analogs which comprise one or more modified sugar moieties, phosphate moieties and/or nucleobases are well known to those of skill in the art. Chimeric oligonucleotides, for example, an oligonucleotide that contains both phosphodiester and phosphorothioate linkages are also encompassed by the present invention.

A modified nucleotide can be produced by a chemical modification either prior to, during, or subsequent to incorporation into an oligonucleotide, for example, using methods that are well known in the art. For example, a modified nucleotide can be produced by incorporating a modified nucleoside triphosphate into a nucleic acid polymer chain during an amplification reaction (e.g., using a polymerase chain reaction (PCR)). Such modified nucleotides, in addition to those already described, include but are not limited to, dideoxynucleotides, biotinylated nucleotides, amine-modified nucleotides, alkylated nucleotides, fluorophore-labeled nucleotides, radiolabeled nucleotides, phosphorothioates, phosphoramidites, phosphites, ring atom-modified derivatives and the like. Oligonucleotides containing multiple modified nucleotides and/or any combination of modified nucleotides are also encompassed by the invention. Oligonucleotides further encompass oligonucleotide polymers which possess a modified backbone, for example, protein-nucleic acids (PNAs) or PNA hybrids. Methods for producing such modified oligonucleotides or oligonucleotide polymers are well known to those of skill in the art.

In one embodiment, the methods of the invention are used to separate a synthetic oligonucleotide from an impurity. In other embodiments, the synthetic oligonucleotide to be separated has a preferred length, for example, from about 2 to about 100 nucleotides, from about 2 to about 75 nucleotides, or from about 4 to about 50 nucleotides. Many synthetic oligonucleotides of current therapeutic interest comprise from 8 to about 40 nucleotides. Thus, in a preferred embodiment, the oligonucleotide to be separated has a length of from about 8 to about 40 nucleotides.

Numerous strategies exist for obtaining oligonucleotides. Naturally-occurring oligonucleotides can be obtained from various biological materials including but not limited to, organisms, tissues and/or cells from veterinary or human clinical test samples (e.g., test samples collected for diagnostic and/or prognostic purposes). Methods for obtaining such naturally occurring oligonucleotides are well known in the art. For example, cells can be lysed and the resulting lysate can be processed using techniques familiar to one of skill in the art to obtain an aqueous solution of nucleic acid (e.g., DNA and/or RNA) (see, for example, Ausebel, F., et al., Current Protocols in Molecular Biology, Wiley, New York (1988); Maniatis, et al., Molecular Cloning: A Laboratory Manual, Cold Spring Harbor Laboratory, Cold Spring Harbor, N.Y. (1982)).

Alternatively, oligonucleotides, either naturally-occurring or synthetic, can be produced using biological methods, chemical methods or a combination of biological and chemical methods. For example, oligonucleotides can be generated biologically using recombinant nucleic acid methodology, artificial recombination (e.g., the polymerase chain reaction (PCR)) or various cloning strategies (e.g., those that employ vectors and/or restriction enzymes). Oligonucleotides can also be generated chemically, for example, using an automated nucleic acid synthesizer or other known chemical synthesis method. Today, the vast majority of oligonucleotides are produced using an automated synthesizer. Typically, an oligonucleotide is synthesized 3′ to 5′, by reacting, step-wise and in a predetermined order, 5′-protected nucleotides (activated at their respective phosphate group) with the deprotected 5′-position in a terminal nucleotide residue of a growing oligonucleotide chain, which is itself attached to a solid support. The most popular protecting groups for the 5′-position have been strongly hydrophobic, for example, 5′-O-trityl protecting groups (e.g., 5′-O-dimethoxytrityl).

Oligonucleotides can also be subjected to various molecular biological and/or separation techniques, prior to and/or subsequent to, being utilized in the methods of the invention. Examples of such separation techniques include, but are not limited to, affinity separation (e.g., nucleic acid hybridization), electrophoretic separation (e.g., using size-fractionation agarose or polyacrylamide gels) and/or chromatographic separation (e.g., HPLC, ion exchange chromatography, reverse-phase chromatography) (see, for example, Ausebel, F., et al., Current Protocols in Molecular Biology, Wiley, New York (1988); Maniatis, et al., Molecular Cloning: A Laboratory Manual, Cold Spring Harbor Laboratory, Cold Spring Harbor, N.Y. (1982)). The methods of the present invention can also be utilized in combination with other separation processes (for example, anion exchange chromatography, reverse-phase chromatography).

In the methods of the invention, the oligonucleotide is present in a mixture. Mixtures comprising oligonucleotides include solutions which comprise a target oligonucleotide. Such mixtures include samples which contain naturally-occurring oligonucleotides (e.g., samples containing nucleic acids found in a living or dead naturally-occurring or artificially-grown unicellular or multicellular organism). Alternatively, the mixtures can include samples which contain synthetic oligonucleotides.

In one embodiment, the mixture comprising the target oligonucleotide further comprises one or more oligonucleotides having a shorter length than the target oligonucleotide. In a preferred embodiment, the mixture is a synthetic mixture of oligonucleotides, for example, a synthetic mixture obtained using an automated synthesizer. In this embodiment, the mixture, in addition to comprising the target oligonucleotide, further comprises truncated failure sequences. Other mixtures which can contain oligonucleotides include but are not limited to, buffered solutions, e.g., Tris-based solutions, MOPS-based solutions, HEPES-based solutions, acetate-based solutions and phosphate-based solutions. Mixtures which comprise solutions typically used in chemical or biological applications are also encompassed. Such mixtures include samples obtained in synthesizing a target oligonucleotide (e.g., samples comprising a PCR product, samples obtained from an automated synthesizer), samples obtained in sequencing a target oligonucleotide or samples obtained in separating an oligonucleotide (e.g., samples obtained from a chromatographic separation). Mixtures which contain a combination of more than one type of oligonucleotide (e.g., a mixture comprising both naturally-occurring and synthetic oligonucleotides) are also encompassed.

The methods of the invention separate a target oligonucleotide from an impurity. An impurity, as defined herein, is a composition which possesses a different molecular structure than the target oligonucleotide. Separation of the impurity from the target oligonucleotide can be partial or substantially complete. In certain embodiments, the impurity to be separated from the target oligonucleotide comprises one or more oligonucleotides having a shorter length than the target oligonucleotide. In a preferred embodiment, the impurity to be separated from the target oligonucleotide comprises one or more failure sequences generated during the synthesis of the target oligonucleotide. Such failure sequences are generated because in the production of synthetic oligonucleotides, during each stepwise addition of a monomer to the nascent oligonucleotide chain, approximately 1-2% of the coupling reactions may fail (i.e., no monomer addition occurs). Consequently, the resulting products are generally a heterogeneous mixture of oligonucleotides of varying length where the amount of undesired impurities or contaminants (e.g., failure sequences) is proportional to the length of the desired product and overall yield of the synthesis. For most downstream applications, it is often necessary to separate the target oligonucleotide from such failure sequences.

In further embodiments, the impurity to be separated from the target oligonucleotide comprises a salt (i.e. an ionic salt), particularly a metal salt. Salts are common impurities which are often found in samples containing a target oligonucleotide, for example, in samples obtained using anion exchange chromatography. The presence of salts in a sample containing a target oligonucleotide typically necessitates one or more subsequent separation or desalting steps in order to remove the salts prior to utilizing the target oligonucleotide. As described herein, the methods of the invention are advantageous in that they do not require a salt gradient to elute the oligonucleotide and therefore may not require subsequent desalting steps. Accordingly, the methods of the present invention can be employed to achieve a substantial reduction in the concentrations of salt impurities. Salts that can be separated from an oligonucleotide using the methods of the invention include, but are not limited to, NaCl, KCl, MgCl₂, CaCl₂, NaHCO₃, Na₂CO₃, NaOH, NH₄Cl, NaOC(O)CH₃ and NaClO₄. Other salts which can be separated using the methods of the invention are known to those of skill in the art.

When a salt is present in a mixture containing the target oligonucleotide, the salt can be separated from the target oligonucleotide by two mechanisms. If the salt does not bind to the target oligonucleotide or the anion exchange composition, it will simply pass through the titratable anion exchange composition and therefore will be separated from the target oligonucleotide which binds and is subsequently eluted at a higher pH. Alternatively, if the salt binds to the target oligonucleotide or the anion exchange composition, it can be removed by washing the anion exchange composition with target oligonucleotide bound thereon, with a suitable washing solution prior to eluting the target oligonucleotide. Thus, in one embodiment of the invention, the anion exchange composition with target oligonucleotide bound thereon, is exposed to one or more washing steps prior to elution. Washing solutions that are suitable for the methods of the invention are those that have a suitable pH and can remove impurities without causing the target oligonucleotide to release from the titratable anion exchange composition. Such washing solutions include but are not limited to, water and buffered solutions (e.g., Tris-based solutions, MOPS-based solutions, HEPES-based solutions, acetate-based solutions, phosphate-based solutions). Other suitable solutions which can be used to wash the anion exchange composition with oligonucleotide bound thereon are well known in the art.

The methods of the invention utilize a titratable anion exchange composition to purify nucleic acids. As described herein, an anion exchange composition is a composition which contains positive charge and displaceable ions (counterions) of negative charge. As such, an anion exchange composition is capable of binding molecules of negative charge (for example, oligonucleotides) and thereby displacing the negatively-charged counterions. The anion exchanger compositions of the present invention are preferably solid compositions.

The anion exchange compositions used in the present invention are titratable. As used herein, a “titratable anion exchange composition” is an anion exchange composition which is capable of losing positive charge as pH increases. Preferably, the titratable anion exchange compositions used in the present invention lose positive charge as the pH increases from about 7.0 to about 12.0. Titratable anion exchange compositions include compositions comprising a primary amine, a secondary amine or a tertiary amine. Suitable titratable anion exchange compositions include anion exchange compositions comprising polyimizadole, polyhistidine, polylysine, polyethyleneimine, polypropyleneimine, modified polyethyleneimine and poly(ethylene-imine/oxyethylene). In a preferred embodiment, the titratable anion exchange composition is polyethyleneimine, which loses positive charge as the pH increases from about 8 to about 11.

Without wishing to be bound to a particular theory, it is thought that the titratable anion exchange compositions used in the invention, possess atoms (e.g., nitrogen atoms) which are protonated and therefore positively charged at neutral and low pH. For example, polyethyleneimine possesses nitrogen atoms which are positively charged at a pH of about 5-8 (or lower pH). As the pH of the eluting solution is gradually increased, the nitrogen atoms of polyethyleneimine begin to become deprotonated. Thus, for example, in separating a target oligonucleotide from failure sequences, the use of polyethyleneimine allows the failure sequences, which are shorter (and therefore have less negative charge) than the target oligonucleotide, to release from the polyethyleneimine at a lower pH than the longer, more negatively-charged target oligonucleotide. As the pH increases, more and more of the nitrogen atoms of polyethyleneimine become deprotonated which allows the longer failure sequences and eventually the target oligonucleotide to release. Finally, at higher pH, all of the nitrogen atoms of polyethyleneimine become deprotonated and therefore cannot retain negatively-charged oligonucleotides.

Therefore, the use of a titratable anion exchange composition (e.g., polyethyleneimine) eliminates the necessity of utilizing a salt gradient to elute bound oligonucleotides and provides a more rapid, efficient and economical method of oligonucleotide separation.

In one embodiment, the titratable anion exchange composition is conjugated to a support. Conjugation of a titratable anion exchange composition to a support can be by methods that are known in the art (e.g. covalently bonding, optionally via a linker group, such as a divinyl sulphone linker or an epichlorohydrin linker). Preferably, conjugation is performed such that the fixed positive charges and displaceable ions of negative charge (counterions) of the titratable anion exchange composition are accessible to the oligonucleotide (i.e., are surface exposed). Suitable supports include solid or semi-solid supports, for example, synthetic polymer supports, such as silica (e.g., silica gel), polysaccharides, synthetic polyolefins including polystyrenes (e.g., styrene-divinylbenzene copolymer), polyethylenes, polypropylenes, polyacrylics (e.g., polyacrylamides, polyacrylates) and agaroses, for example the agaroses commercially available under the trade name Sepharose. Other suitable supports known in the art are also encompassed by the invention. In a preferred embodiment, the titratable anion exchange composition is polyethyleneimine-derivatized silica gel or polyethyleneimine-derivatized styrene-divinyl benzene copolymer. In another, embodiment, the titratable anion exchange composition is not conjugated to a support. Rather, the solid titratable anion exchange composition itself acts as a support to which the oligonucleotide can be bound and released.

In certain embodiments of the present invention, good results have been achieved by the use of the polyethyleneinine-derivatized silica titratable anion exchange composition commercially available from Millipore under the trade name Matrex Silica, for example Matrex Silica PEI-300 and Matrex Silica PEI-1000.

The concentration and amount of titratable anion exchange composition used in the methods of the invention depend on a number of factors, e.g., the quantity and nature of the mixture comprising the oligonucleotide, the concentration and nature of the oligonucleotide in the mixture and the concentration and nature of the impurities in the mixture. One of skill in the art can easily determine an appropriate amount of titratable anion exchange composition which is suitable for binding the target oligonucleotide, without having to perform undue experimentation.

Similarly, one of skill in the art can determine appropriate parameters for conducting the separation, without having to perform undue experimentation. For example, for relatively pure mixtures comprising the target oligonucleotide or for mixtures comprising impurities which are structurally quite different than the target oligonucleotide, the separation can proceed relatively quickly, e.g., using an increased flow rate. Alternatively, for highly heterogeneous mixtures or mixtures containing an impurity that is similar to the target oligonucleotide, it may be desirable to increase the resolution by slowing down the separation (e.g., using a decreased flow rate) and/or utilizing additional titratable anion exchange composition.

Another important variable which can be altered to achieve suitable separation is the rate of change in pH of the eluting solution. Again, depending on the nature of the separation, the person of skill in the art will be able to determine a suitable pH elution profile (e.g., continuous pH gradient, step-wise pH gradient, rapid rate of change in pH, slow rate of change in pH).

A wide variety of eluting solutions which increase in pH can be used in the methods of the invention including solutions which are suitably buffered to achieve the desired pH, e.g., tris-based solutions, MOPS-based solutions, HEPES-based solutions, acetate-based solutions, phosphate-based solutions. Other solutions which are typically used in anion exchange separations and are known in the art are also encompassed. The eluting solution can also contain one or more ionic salts, e.g., NaCl, KCl, MgCl₂, CaCl₂, NaHCO₃, Na₂CO₃, NaOH, NH₄Cl, NaOH and the like, however, as described herein, the eluting solutions do not utilize a salt gradient. The volume of the eluting solution used in the methods of the present should be sufficient to saturate the titratable anion exchange composition.

In a preferred embodiment, the eluting solution which is passed through the titratable anion exchange composition with the target oligonucleotide bound thereon, increases in pH over time but does not substantially increase its salt concentration over the same period of time. As defined herein, the lack of a “substantial increase” in salt concentration means that any increase in salt concentration which occurs does not significantly affect the elution of the target oligonucleotide from the titratable anion exchange composition.

In another embodiment, the eluting solutions used in the present invention have a substantially constant salt concentration. As used herein, a “substantially constant” salt concentration means that any variations in salt concentration (increase or decrease) are small enough that they do not significantly affect elution of the target oligonucleotide from the titratable anion exchange composition. Alternatively, the salt concentration of the eluting solution can decrease over time. In another embodiment, the eluting solution is substantially free of metal salts. As used herein, “substantially free of metal salts” refers to the fact that the solution contains an insubstantial amount of metal salts (i.e., no metal salts or so little metal salts as to not affect downstream applications which utilize the oligonucleotide). One important aspect of invention is that unlike typical anion exchange separations of oligonucleotides, the methods of the present invention utilize a pH gradient and not a salt gradient for elution.

The eluting solutions used in the present invention include solutions that increase in pH in a step-wise manner as well as solutions that increase in pH in a continuous manner. In one embodiment, the solution used to elute the target oligonucleotide increases in pH in a linear manner over time. In another embodiment, the solution used to elute the target oligonucleotide increases from a pH of about 8 to a pH of about 11. Preferably, in this embodiment, the titratable anion exchange composition which is utilized comprises polyethyleneimine. The eluting solutions can also comprise one or more buffering agents for suitably altering the pH. In one embodiment, the eluting solution comprises one or more of NaHCO₃, Na₂CO₃, NaOH, NH₄OH and/or NH₄HCO₃. In a preferred embodiment, the eluting solution is free from metal salts, and advantageously comprises salts which are volatile on drying, such as NH₄HCO₃ and especially NH₄OH. Although the elution rate is generally not critical, the solution used to elute the target oligonucleotide is generally administered at a flow rate of about 350 to 550 cm/hr and more commonly at a rate of between about 420 to 450 cm/hr.

It will be recognised that the eluting solutions which comprise salts have a salt concentration substantially lower than the salt concentrations employed in salt-gradient elution. Often, the concentration of the salts in a pH gradient eluting solution is no more than 0.1M, such as from about 0.01 to about 0.07M, for example about 0.05M. In many embodiments, the overall increase in pH with the pH gradient does not increase the salt concentration in the eluting solution by more than about 100%. In certain particular embodiments, the salt concentration may remain substantially constant or even decrease. The salt concentration of the eluting solution commonly does not exceed 0.2M, and preferably is no more than 0.1M. This contrasts with the use of a salt gradient, where the salt concentrations are typically at least about 0.5M, commonly increasing to 2M or even higher, such as 3 to 4M.

The methods of the invention can be used for the separation of 5′-O-protected oligonucleotides. In certain embodiments, the 5′-O-protecting group can be cleaved from the target oligonucleotide while it is still bound to the titratable anion exchange composition. Suitable 5′-O-protecting groups are known in the art and include, for example, substituted or unsubstituted trityl groups (e.g., 4,4′-dimethoxytrityl (DMT)). In a preferred embodiment, the methods of the invention are used for the separation of a 5′-O-trityl protected oligonucleotide (e.g., a 5′-O-dimethoxytrityl protected oligonucleotide). Cleavage of the protecting group from the bound oligonucleotide (i.e., prior to eluting off of the titratable anion exchange composition) can be achieved by passing a suitable reagent through the titratable anion exchange composition. For cleavage of a trityl protecting group from a target oligonucleotide, a suitable reagent is a sufficient amount of an acidic solution. In many embodiments, a trityl protecting group (e.g., 5′-O-DMT) can be cleaved from the target oligonucleotide using aqueous acetic acid, for example 20%-80% v/v aqueous acetic acid.

The methods of the present invention are advantageous in that they avoid the more laborious and time-consuming conventional separation steps which are typically required to separate or purify oligonucleotides. In eliminating the necessity of utilizing a salt gradient in anion exchange separations, the methods of the invention reduce the number of steps which is typically required to separate oligonucleotides. Thus, the present invention provides a rapid, simplified, more efficient and less expensive method for large-scale purification of oligonucleotides. Moreover, in eliminating the use of high concentrations of salt in eluting solutions, the methods of the invention decrease damage to stainless steel and/or moving parts of chromatographs and other anion exchange separation machinery (e.g., HPLC pumps, fittings, valves, columns, tubing).

The invention will be further described by the following non-limiting examples. The teachings of all publications cited herein are incorporated herein by reference in their entirety.

### EXAMPLES

### Example 1

### Purification of an Oligonucelotide Prepared by Solid Phase Synthesis

The following equipment and reagents are referred to herein and for convenience will be listed once with the pertinent information. Unless otherwise indicated, all equipment and reagents were used as directed in the manufacturer's instructions. Further, unless otherwise indicated, other similar equipment and reagents can be substituted, as is well known to those skilled in the art.

Reagents

Polyethyleneimine-derivatized silica gel (Matrex Ion Exchange Silica PEI-300-15; product number S674 (Millipore, Bedford, Mass.);
Buffer A; 50 mM NaHCO₃ solution; pH 8.2;
Buffer B; 50 mM NaHCO₃/Na₂CO₃ solution; pH adjusted to 11.1 with 0.1 M NaOH;
Buffer C, 0.1 NaOH solution;
Buffer D; 0.1 M Tris, 2 M NaCl solution; pH 7.5

The oligonucleotide TCG-TCG-TGT-TTT-CTA-TTT-TCG-UTT (SEQ ID NO. 1) was synthesized using solid support and phosphoramidite chemistry. The 5′-O-trityl protecting group was removed at the end of oligonucleotide synthesis using 3% dichloroacetic acid in methylene chloride. The oligonucleotide was then released from the support and protecting groups were removed with concentrated ammonium hydroxide solution. The solution containing the target oligonucleotide was then adjusted to pH 7.5 by adding 1.0 M phosphoric acid solution (total oligonucleotide was 121,950 OD).

Separation of Oligonucleotide

A slurry of PEI-derivatized silica gel (100 mL) was made in Buffer D and was packed in a glass column at a flow rate of 8 mL/minute. The column was subsequently washed with Buffer C, Buffer D and Buffer A until the resulting washes attained pH 8.2. The column was equilibrated by washing it with 4 column volumes of Buffer A at a flow rate of 35 mL/minute.

The sample containing the target oligonucleotide was loaded on to the column at a constant flow rate of 35 ml/minute. After the sample was loaded, the column was washed with 4 column volumes of Buffer A. The target oligonucleotide was separated using a pH gradient (pH 8.2 to 11.1) which was obtained by using Buffer A and Buffer B (0 to 80% Buffer B in 20 column volumes at a flow rate of 435 cm/hr). The fractions were collected and assayed for the desired oligonucleotide using analytical anion exchange HPLC. Fractions with purity greater than 90% were pooled. Concentration of the oligonucleotide product and desalting were either carried out using UF membrane filtration or by binding to PEI-derivatized silica gel. The oligonucleotide product was then lyophilized.

Regeneration of the Column

After separation of the target oligonucleotide, the column was regenerated by washing it with Buffer C (1 column volume), Buffer D (2 column volumes) and finally with Buffer A (until washes attained pH less than 8.5). The column is then suitable for additional separations.

### Example 2

### Desalting of an Oligonucleotide Prepared by Solid Phase Synthesis and Purified by Ion Exchange Chromatography

Reagents

Polyethyleneimine-derivatized silica gel (Matrex Ion Exchange Silica PEI-300-15; product number S674 (Millipore, Bedford, Mass.);
Buffer A; 0.25 M NH₄HHCO₃ solution; pH 7.5;
Buffer B; 0.1 M NH₄OH;
Buffer C; MILLI-Q Water
Buffer D; 0.1 M NH₄OH, 2 M NaCl solution; pH 7.5

A slurry of PEI-derivatized silica gel (100 mL) was made in Buffer D and was packed in a glass column at a flow rate of 8 mL/minute. The column was subsequently washed with two column volumes of Buffer D. The column was then re-equilibrated with Buffer A until the pH was less than 8.

The oligonucleotide TCG-TCG-TGT-TTT-CTA-TTT-TCG-UTT (SEQ ID NO. 1) was synthesized as described in Example 1. After synthesis, the target oligonucleotide was separated from the by-products by ion exchange chromatography using standard means, resulting in solution with a pH 12 that contained the purified oligonucleotide and about 1.5-2.0 M sodium chloride. The pH of this solution was then adjusted to 7.5 with 1.0 M acetic acid. The sample was then loaded onto the column at a flow rate of 8 mL/minute (1000 OD/mL resin).

The loaded column was first washed with five column volumes of Buffer A, followed by washing with Buffer C until the conductivity was less than 0.01 millisiemons/cm. The oligonucleotide was then eluted from the column with Buffer B. After elution, the column was regenerated by washing with two column volumes of Buffer A.

### Example 3

An 18-mer fully phosphorothioated deoxyribonucleotide containing 66% full length product (FLP) was purified 5′-dimethoxytrityl on using the method of Example 1, except that Buffer A had a pH of 6.0. Analysis of the purified, eluted nucleotide showed a product purity of >94% FLP. Comparable results were also achieved when a PEI-derivatised polystyrene bead was employed as the titratable anion-exchange support.

While this invention has been particularly shown and described with references to preferred embodiments thereof, it will be understood by those skilled in the art that various changes in form and details may be made therein without departing from the scope of the invention encompassed by the appended claims.

1. A method of separating a target oligonucleotide from an impurity, in a mixture comprising said target oligonucleotide and said impurity, using a titratable anion exchange composition, comprising the steps:
a) binding said target oligonucleotide to said titratable anion exchange composition;
b) passing a solution through said titratable anion exchange composition with target oligonucleotide bound thereon, wherein said solution increases in pH over time; and
c) eluting said target oligonucleotide, wherein said impurity elutes at a different pH than said target oligonucleotide.

2. The method of claim 1 wherein said titratable anion exchange composition comprises a primary amine, a secondary amine or a tertiary amine.

3. The method of claim 1 or claim 2, wherein said titratable anion exchange composition comprises polyethyleneimine, polyimizadole, polyhistidine or polylysine.

4. The method of claim 1, wherein said solution in b) is substantially free of metal salts.

5. The method according to claim 1, wherein the solution in b) does not substantially increase its salt concentration over time.

6. The method of claim 1, wherein said titratable anion exchange composition is conjugated to a support.

7. The method of claim 6, wherein said support is a synthetic polymer.

8. The method of claim 0.7, wherein said synthetic polymer is selected from the group consisting of silica gel, a polysaccharide, a styrene-divinyl benzene copolymer, a polyethylene, a polypropylene, a polyacrylic and an agarose.

9. The method of claim 8, wherein said titratable anion exchange composition is polyethyleneimine-derivatized silica gel or a polyethyleneimine-derivatized styrene-divinyl benzene copolymer.

10. The method of claim 1, wherein said target oligonucleotide is a synthetic oligonucleotide.

11. The method of claim 10, wherein said synthetic oligonucleotide is selected from the group consisting of a phosphorothioate, a phosphorodithioate, a methyl phosphonate and a phosphoramidate.

12. The method of claim 1, wherein binding of said target oligonucleotide with said titratable anion exchange composition occurs at a pH between 5 and 8.

13. The method of claim 1, wherein said solution in b) increases in pH in a linear manner over time.

14. The method of claim 1, wherein said solution in b) increases from a pH of about 8 to a pH of about 11.

15. The method of claim 1, wherein said solution in b) comprises one or more of NH₄HCO₃ and/or NH₄OH.

16. The method of claim 1, wherein said target oligonucleotide has a length from about 8 to about 40 nucleotides.

17. The method of claim 1, wherein said impurity is one or more oligonucleotides having a shorter length than said target oligonucleotide, and wherein said impurity elutes at a lower pH than said target oligonucleotide.

18. The method of claim 17, wherein said impurity is one or more failure sequences.

19. The method of claim 1, wherein said impurity is a metal salt.

20. The method of claim 1, wherein said target oligonucleotide is 5′-O-protected.

21. The method of claim 20, wherein said target oligonucleotide is 5′-O-trityl protected.

22. The method of claim 21, further comprising a step of passing through said titratable anion exchange composition a sufficient amount of an acidic solution to cleave said 5′-O-trityl protecting group from said target oligonucleotide prior to eluting said target oligonucleotide.

23. The method of claim 22 wherein said acidic solution comprises aqueous acetic acid.

24. The method of claim 1, wherein said solution in b) has a volume which is less than the volume of the mixture comprising said target oligonucleotide and impurity, thereby increasing the concentration of said target oligonucleotide.

25. The method of claim 1, further comprising one or more washing steps prior to eluting said target oligonucleotide.

26. The method of claim 21, wherein said target oligonucleotide is 5′-O-dimethoxy-trityl protected.

27. The method of claim 1, wherein said titratable anion exchange composition comprises polyethyleneimine, polyimizadole, polyhistidine or polylysine conjugated to a synthetic polymer support; said solution in b) is substantially free of metal salts, does not substantially increase its salt concentration over time, increases from a pH of about 8 to a pH of about 11 and comprises one or more of NH₄HCO₃ and/or NH₄OH.
